(19) United States
(12) Patent Application Publication
(10) Pub. No.: US 2018/0344530 A1
(43) Pub. Date: Dec. 6, 2018

(54) PRESSURE RELIEF DEVICE FOR EARS

(71) Applicant: ALI A.A.J. SHAMMOH, SAFAT (KW)

(72) Inventor: ALI A.A.J. SHAMMOH, SAFAT (KW)

(21) Appl. No.: 15/978,805

(22) Filed: May 14, 2018

Related U.S. Application Data
(60) Provisional application No. 62/515,482, filed on Jun. 5, 2017.

(51) Int. Cl. A61F 11/06; A61F 7/02

(52) U.S. Cl. CPC A61F 11/06; A61F 2007/0005; A61F 7/02

(57) ABSTRACT

The pressure relief device for ears includes first and second ear assemblies, corresponding to first and second ears of a user, and a resilient headband. Each of the first and second ear assemblies includes a hollow vapor chamber, an earpiece, a container and an absorbent pad. The hollow vapor chamber includes an upper portion and an open lower portion. An open internal channel is formed through the hollow vapor chamber. The earpiece has an opening formed therethrough and is adapted for engaging the corresponding ear of the user. The earpiece is secured to the upper portion of the hollow vapor chamber, and the opening is in open fluid communication with the open internal channel. The container is releasably secured to the open lower portion of the hollow vapor chamber and is in open fluid communication with the open internal channel. The absorbent pad is removably received in the container.

### CROSS-REFERENCE TO RELATED APPLICATION

This application claims the benefit of U.S. Provisional Patent Application Ser. No. 62/515,482, filed on Jun. 5, 2017.

### BACKGROUND

### 1. FIELD

The disclosure of the present patent application relates to ear pressure reduction, and particularly to a pressure relief device for ears that can be used for relieving ear pressure build-up.

### 2. DESCRIPTION OF THE RELATED ART

An imbalance between the pressure in the middle ear and ambient pressure can result in the sensation of having one's ears clogged. Individuals typically suffer from clogged ears as a result of a colds, allergies, or descent in an aircraft from high altitude. If not treated or naturally relieved, clogged ears may lead to chronic traumatic inflammation of the middle ear, also known as aerotitis media, aviator's ear, otic barotrauma, barotitis media, and aviation otitis. People who suffer from clogged ears typically try to relieve any ear discomfort by yawning, swallowing, chewing gum, or closing the nostrils and blowing lightly to attempt to equalize the pressure in the middle ear. These methods, however, yield unpredictable results and, in the case of the latter technique, can accidentally cause injury. Thus, a pressure relief device for ears solving the aforementioned problems is desired.

### SUMMARY

The pressure relief device for ears includes first and second ear assemblies corresponding to first and second ears of a user. Each of the first and second ear assemblies includes a hollow vapor chamber, an earpiece, a container and an absorbent pad. The hollow vapor chamber includes an upper portion and an open lower portion. An open internal channel is formed through the hollow vapor chamber. The earpiece has an opening formed therethrough and is adapted for engaging the corresponding ear of the user. The earpiece is secured to the upper portion of the hollow vapor chamber, and the opening is in open fluid communication with the open internal channel formed through the hollow vapor chamber. The container is releasably secured to the open lower portion of the hollow vapor chamber and is in open fluid communication with the open internal channel formed through the hollow vapor chamber. The absorbent pad is removably received in the container.

A resilient headband extends between, and supports, the first and second ear assemblies. In use, a hot liquid, such as hot water, is absorbed by the absorbent pad of each of the first and second ear assemblies. Water vapor evaporating therefrom flows through the open internal channel formed through the hollow vapor chamber of each of the first and second ear assemblies and into ear canals of the user in order to relive pressure build-up in the user's ears.

These and other features of the present disclosure will become readily apparent upon further review of the following specification and drawings.

### BRIEF DESCRIPTION OF THE DRAWINGS

FIG. 1 is a perspective view of a pressure relief device for ears.

FIG. 2 is a partially exploded perspective view of the pressure relief device for ears.

### Similar reference characters denote corresponding features consistently throughout the attached drawings.

### DETAILED DESCRIPTION OF THE PREFERRED EMBODIMENTS

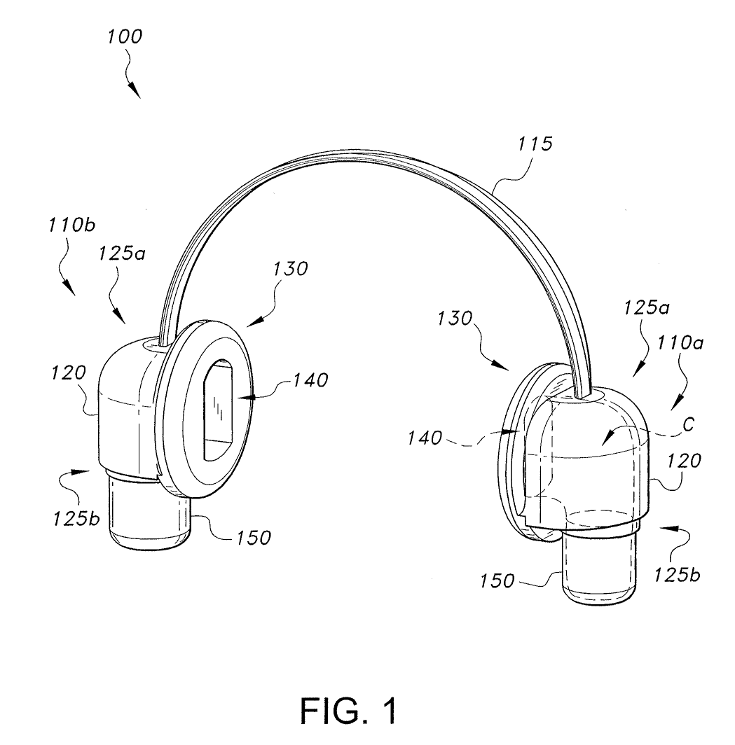

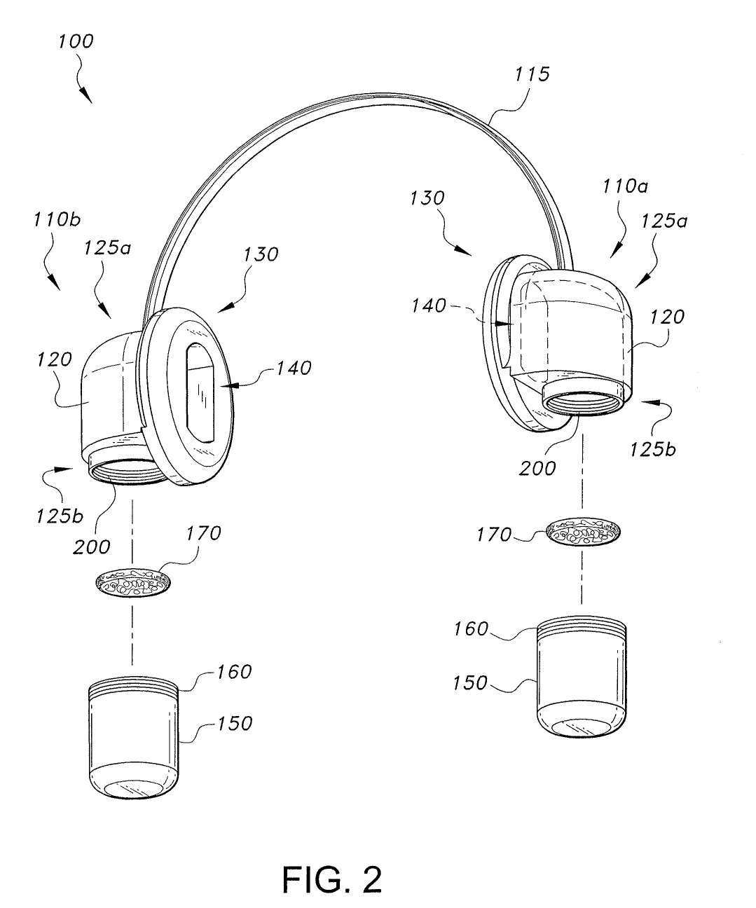

Referring now to FIGS. 1 and 2, the pressure relief device for ears 100 is configured for relieving ear pressure build-up. The pressure relief device for ears 100 includes a right ear assembly 110a and a left ear assembly 110b. As shown, right ear assembly 110a is connected to left ear assembly 110b by an elastic headband 115 or the like. Each ear assembly 110a, 110b includes a vapor chamber 120, which has an upper portion 125a connected to the headband 115 and an open lower portion 125b. As shown in FIG. 2, the open lower portion 125b of the vapor chamber 120 of each assembly 110a, 110b is preferably internally threaded with threads 200.

An earpiece 130 is connected to the vapor chamber 120 of each ear assembly 110a, 110b. The earpiece 130 of each ear assembly 110a, 110b may have a generally circular or oval configuration on one side, as is conventionally known, and is adapted to engage a corresponding ear of the user. The earpiece 130 of each ear assembly 110a, 110b can be formed from any suitable material, such as plastic or the like, and may be configured to cushion the user's ears. The earpiece 130 of each assembly 110a, 110b has an opening 140 formed therethrough, as shown. Opening 140 may be large enough to accommodate the user's ear, or earpiece 130 may be sized such that headband 115 resiliently presses earpiece 130 against the outer ear, with opening 140 being substantially centrally located with respect to the user's ear. The opening 140 of the earpiece 130 is in open fluid communication with the vapor chamber 120 through a channel C, as illustrated in FIG. 1.

A container 150, such as a cup or the like, is removably connected to the open lower portion 125b of the vapor chamber 120 of each ear assembly 110a, 110b. The container 150 preferably has external threads 160 formed at an open mouth thereof for releasably engaging the internal threads 200 of the open lower portion 125b of the vapor chamber 120. The container 150 is configured for receiving a suitable absorbent material, such as an absorbent disk or pad 170. The absorbent disk or pad 170 may be made of cotton, for example, or any other suitable material suitable for absorption of hot or warm water. Hot or warm water absorbed into absorbent disk or pad 170 provides moist heat, such as heated or warm water vapor, which passes upward through the channel C and into the user's ear canals to relieve pressure on the user's ears.

The headband 115 may be formed from any suitable type of resilient flexible material, such as plastic, so that the pressure relief device for ears can snugly fit a wide variety of different users. Further, it should be understood that the vapor chamber 120 of each ear assembly 110a, 110b may be formed from any suitable material, such as plastic or the like, and may have a generally hollow configuration to facilitate the transfer of vapor from the absorbent pad 170 through the channel C and into the user's ears to relieve the pressure on the ear(s).

In use, a person suffering from ear pressure build-up initially immerses the two absorbent pads 170 (i.e., one for each container 150) in hot or warm water. Once the absorbent pads 170 have been saturated with the hot or warm water, the absorbent pads 170 are removably inserted into the container 150 of each ear assembly 110a, 110b. The container 150 of each ear assembly 110a, 110b is then attached to the open lower portion 125b of the vacuum chamber 120 of each assembly 110a, 110b. The head band 115 of the pressure relief device for ears 100 is then fitted over the user's head.

It is to be understood that the pressure relief device for ears is not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. A pressure relief device for ears, comprising:
first and second ear assemblies, wherein each of the first and second ear assemblies comprises:
a hollow vapor chamber having an upper portion, an open lower portion, and an open internal channel formed between the upper portion and the lower portion;
an earpiece adapted for engaging an ear of a user, the earpiece having an opening formed therethrough and being secured to the upper portion of the hollow vapor chamber, the opening being in open fluid communication with the open internal channel formed through the hollow vapor chamber;
a container releasably secured to the open lower portion of the hollow vapor chamber, the container being in open fluid communication with the open internal channel formed through the hollow vapor chamber; and
an absorbent material removably received in the container; and
a resilient headband extending between, and supporting, the first and second ear assemblies, whereby a hot liquid may be absorbed by the absorbent material of each of the first and second ear assemblies, such that water vapor evaporating therefrom may flow through the open internal channel formed through the hollow vapor chamber of each of the first and second ear assemblies and into ear canals of the user.

2. The pressure relief device for ears as recited in claim 1, wherein each opening of each said earpiece is generally oval.

3. The pressure relief device for ears as recited in claim 1, wherein an open upper end of the container of each of the first and second ear assemblies is threadedly attached to the open lower portion of the hollow vapor chamber thereof.

4. The pressure relief device for ears as recited in claim 1, wherein the absorbent material comprises an absorbent pad.

5. The pressure relief device for ears as recited in claim 1, wherein the absorbent material comprises cotton.

6. A pressure relief device for ears, comprising:
first and second ear assemblies, wherein each of the first and second ear assemblies comprises:
a hollow vapor chamber having an upper portion, an open lower portion, and an an open internal channel extending between the upper portion and the lower portion;
an earpiece adapted for engaging an ear of a user, the earpiece having an opening formed therethrough and being secured to the upper portion of the hollow vapor chamber, the opening being in open fluid communication with the open internal channel formed through the hollow vapor chamber;
a container threadedly attached to the open lower portion of the hollow vapor chamber, the container being in open fluid communication with the open internal channel formed through the hollow vapor chamber; and
an absorbent pad formed from an absorbent material, the absorbent pad being removably received in the container; and
a resilient headband extending between, and supporting, the first and second ear assemblies, whereby a hot liquid may be absorbed by the absorbent material of each of the first and second ear assemblies, such that water vapor evaporating therefrom may flow through the open internal channel formed through the hollow vapor chamber of each of the first and second ear assemblies and into ear canals of the user.

7. The pressure relief device for ears as recited in claim 6, wherein each opening of each said earpiece is adapted for receiving a corresponding one of the ears of the user.

8. The pressure relief device for ears as recited in claim 6, wherein the absorbent material comprises cotton.
